(19) United States
(12) Patent Application Publication
(10) Pub. No.: US 2008/0208173 A1
(43) Pub. Date: Aug. 28, 2008

(54) Insulin Pump Having Triple Functions Which Enables Efficient Control of Blood Glucose Concentration and Insulin Injection System Comprising the Same

(76) Inventors: Hong-Kyu Lee, Seoul (KR); Hee-Chan Kim, Seoul (KR)

Correspondence Address: HOFFMANN & BARON, LLP, 6900 JERICHO TURNPIKE, SYOSSET, NY, 11791, US

(21) Appl. No.: 11/667,168

(22) PCT Filed: Nov. 4, 2005
(86) PCT No.: PCT/KR05/03704

§ 371 Date: Nov. 30, 2007

(30) Foreign Application Priority Data
Nov. 4, 2004 (KR) 10-2004-0089241

(51) Int. Cl. A61M 5/142

(52) U.S. Cl. 604/890.1

(57) ABSTRACT

The present invention relates to an insulin pump having triple functions for the efficient regulation of a blood glucose concentration and an insulin injection system comprising the same. The insulin pump comprises an insulin syringe into which an insulin is stored, a pump to pressurize the insulin syringe in response to a programmed control signal, a control device that generates the programmed control signal to control the pump, a mode selection button to determine an insulin injection mode, and an operating button to command an insulin injection. The insulin injection mode is comprised of a base mode that continuously infuses the insulin at a basic rate to control a blood glucose concentration at a fasting, a food mode that infuses the insulin at a bulk to control the blood glucose concentration after food intake, and an activation mode that infuses the insulin in pulses for a predetermined time period. Preferably, the insulin pump complementarity cooperates with a physiological saline syringe pump that infuses physiological saline solution immediately after the infusion of the insulin at the activation mode.

### TECHNICAL FIELD

The present invention relates to an insulin pump for the efficient regulation of a blood glucose concentration, more specifically, to an insulin pump having triple functions for the efficient regulation of a blood glucose concentration.

### BACKGROUND ART

FIG. 1 is a graph showing an insulin secretion profile, in which FIG. 1(a) is the profile for a normal man and FIG. 1(b) is for a diabetic patient. As shown in FIG. 1(a), for the normal human being, secretion of the insulin is proportional to glucose level. To the contrary, for the diabetic patents, the insulin is not sufficiently secreted even after the administration of food such that high glucose level is continued for an extended time. In order to prevent collapse of the glucose regulation mechanism, external infusion of the insulin should be properly performed.

According to conventional NPH (Neutral Protamine Hagedorn) therapy, the insulin is externally infused one or two times per day. FIG. 2 shows a blood insulin profile in accordance with the NPH therapy. As shown in FIG. 2, the blood insulin profile of the NPH therapy is significantly different, in terms of time and quantity, with the physiological insulin profile. Specifically, when high insulin level is physiologically required, the NPH therapy does not satisfy such a requirement. In addition, at a normal situation, the NPH therapy shows higher insulin level than required. In the NPH therapy, in order to prevent hypoglycemia, the insulin should be infused as small as possible. Due to irrational profile of the insulin, the NPH therapy develops sometimes various complications. Therefore, in order to achieve enhanced regulation of the blood glucose concentration, the insulin should be administered in a physiological profile.

Insulin pumps have been used in order to supply the insulin in a physiological profile. U.S. Pat. No. 4,282,872 discloses an insulin pump for the delivery of the insulin. According to the document, the insulin pump consists of two modes: one is a base mode that permits continuous administration of the insulin at a basic rate; and the other is a food mode that permits one bulk administration of the insulin after food intake at an amount required for the normal human being.

The insulin pump with the two modes was known to be effective for the control the blood glucose concentration and for the prevention of complications accompanied by the diabetes. The insulin pump aimed at maintaining the glucose level to the normal condition by external injection of the insulin. Even though the insulin pump gave more enhanced effects, there remained unresolved problems. In the normal physiological condition, the insulin secreted from a pancreas is delivered to a kidney such that the kidney is exposed to the high level of the insulin. To the contrary, in the conventional insulin therapy including the insulin pump therapy, whole tissues are exposed to the highly concentrated insulin, while the kidney is exposed to relatively low concentration of the insulin. If this situation continues for an extended time, the function of the kidney is deteriorated. This adversely affects on organs related to the kidney and results in secondary changes. That is, as the quantity of the insulin secreted from the pancreas decreases, other tissues and organs related to the glucose metabolism conform to such a decrease so that they undergo functional changes to comply with the decrease. The conventional insulin pump therapy does not solve such a disadvantage. For instance, external infusion of excess insulin sometimes yields unbalance in the glucose metabolism, and results in side effects. Particularly, for the patients sensitive to the insulin, external infusion of excess insulin causes hypoglycemia, and sometimes results in death. Furthermore, at an abnormal condition, such an abrupt infusion or deficiency of the insulin may result in significant side effects.

Instability of the insulin therapy can be minimized by intermittent, pulsed infusions of the insulin, called as “activated treatment”. Further, the pulsed infusions of the insulin for an extended period are known to increase the processing ability of glucose and lipid by the kidney and prevent complicating diseases accompanied by the diabetes (U.S. Pat. No. 4,826,810). Up to now, the activated treatment was performed with additional, separated pump. In this report, we provide a new system having triple functions to which the activated treatment is integrated to the conventional insulin pump to solve the conventional insulin pump therapy.

### DISCLOSURE OF INVENTION

### Technical Problem

An object of the present invention is to provide an insulin pump having triple functions for the efficient control of the blood glucose concentration.

Another object of the present invention is to provide an insulin injection system into which a physiological saline syringe pump, in addition to the insulin pump, is incorporated for the efficient administration of the insulin at the activated treatment.

### Technical Solution

According to a preferred embodiment of the present invention, there is provided an insulin pump comprising: a) an insulin syringe into which an insulin is stored; b) a pump to pressurize the insulin syringe in response to a programmed control signal; c) a control device that generates the programmed control signal to control the pump; d) a mode selection button to determine an insulin injection mode; and e) an operating button to command an insulin injection, wherein f) the control device is equipped with a memory to store a control program and a controller to generate the programmed control signal under control of the control program, g) the control program is comprised of a base mode that continuously infuses the insulin at a basic rate to control a blood glucose concentration at a fasting, a food mode that infuses the insulin at a bulk to control the blood glucose concentration after food intake, and an activation mode that infuses the insulin in pulses for a predetermined time period, h) mode selection among the three modes is carried out by the mode selection button, i) in response to activation of the operating button, the controller of the control device delivers to the pump the programmed control signal corresponding to the selected mode, and j) with action of the pump, the insulin syringe infuses the insulin in the selected mode.

According to another preferred embodiment of the present invention, there is provided an insulin injection system comprising a physiological saline syringe pump in combination with the insulin pump, wherein the physiological saline syringe pump comprises: a′) a physiological saline syringe into which physiological saline solution is stored; b′) a physiological saline pump to pressurize the physiological saline syringe in response to a programmed control signal; c′) a control device that generates the programmed control signal to control the physiological saline pump; d′) a selection button; e′) a mount for housing the insulin pump; and f′) an access port for the synchronization with the insulin pump. Herein, the infusion of the physiological saline solution is carried out at the activation mode in synchronization with the action of the insulin pump.

### ADVANTAGEOUS EFFECTS

According to the present invention, the insulin can be administered by three modes: a base mode that continuously infuses the insulin at a basic rate to control a blood glucose concentration at a fasting; a food mode that infuses the insulin at a bulk to control the blood glucose concentration after food intake; and an activation mode that infuses the insulin in pulses for a predetermined time period. This enables more improved regulation of the blood glucose concentration, depending on the condition of the patient, sensitivity to the insulin, the kind of the food administered. Further, infusion of the physiological saline solution synchronized with the infusion of the insulin can overcome the limitation of the activated treatment using the conventional insulin pump alone. Therefore, the insulin injection system of the present invention, comprised of integrated combination of the insulin pump with the physiological saline syringe pump, is a new system that extends the advantage of the conventional insulin pump.

### BRIEF DESCRIPTION OF THE DRAWINGS

FIG. 1 is a graph showing an insulin secretion profile, in which (a) is the profile for a normal human being and (b) is for a diabetic patient.

FIG. 2 is a graph showing another blood insulin profile in accordance with the conventional NPH therapy.

FIG. 3 is a graph showing a blood insulin profile obtained from pulsed infusion of the insulin to induce activation of metabolism of the kidney.

FIG. 4 is a block diagram showing a preferred embodiment of the insulin pump, in accordance with the present invention.

FIG. 5 is a block diagram showing a preferred embodiment of the insulin injection system comprising the insulin pump, in accordance with the present invention.

FIG. 6 is a block diagram showing another preferred embodiment of the insulin injection system comprising the insulin pump, in accordance with the present invention.

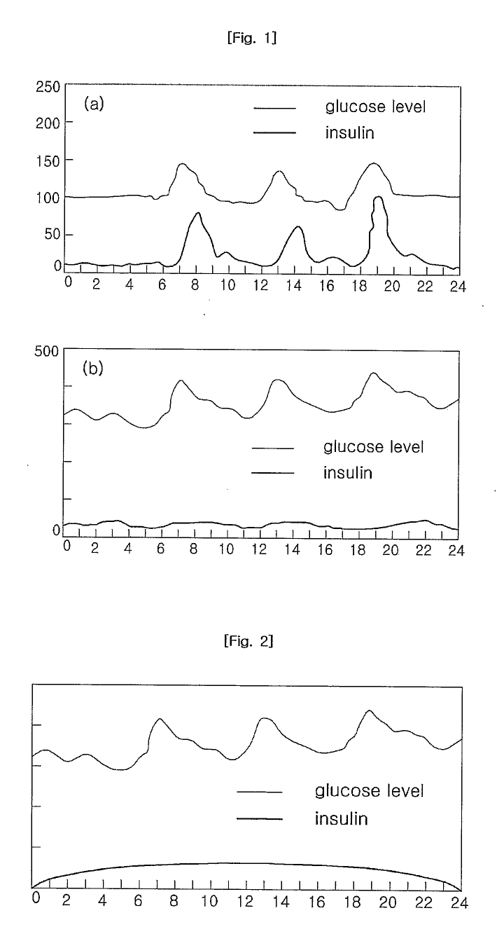

### MODE FOR THE INVENTION

The insulin pump according to the present invention comprises: an insulin syringe into which an insulin is stored; a pump to pressurize the insulin syringe in response to a programmed control signal; a control device that generates the programmed control signal to control the pump; a mode selection button to determine an insulin injection mode; and an operating button to command an insulin injection. Herein, the control device is equipped with a memory to store a control program and a controller to generate the programmed control signal under control of the control program, and the control program is comprised of a base mode that continuously infuses the insulin at a basic rate to control a blood glucose concentration at a fasting, a food mode that infuses the insulin at a bulk to control the blood glucose concentration after food intake (wherein, the base mode and the food mode constitute a maintenance mode), and an activation mode that intravenously infuses the insulin in pulses for a predetermined time period. The mode conversion among the three modes is carried out by the mode selection button, and in response to activation of the operating button, the controller of the control device delivers to the pump the programmed control signal corresponding to the selected mode, and with action of the pump, the insulin syringe infuses the insulin in the selected mode. The maintenance mode consisting of the base mode and the food mode is preferably managed by patients, and the activation mode is by doctors. The access to the activation mode by the patients may be preferably prohibited. The activation mode is used for the activation of the metabolism of the kidney after food intake in order to restore the response to the insulin. For example, the activation mode is weekly applied, under the control of the doctors, to patients suffered from insulin-dependent diabetes mellitus. In a daily activity, the maintenance mode is applied. The infusion of the insulin through the maintenance mode is the same with that of the conventional insulin pump. For the well-trained patients, the activation mode can be performed by themselves under remote control of the doctors, but this is an exceptional case.

In a meanwhile, the food mode is re-executed only after a predetermined time interval has been elapsed. The food mode is to control abrupt increase of the glucose level after food intake, and re-execution is made only after typically 4-8 hours have been elapsed. This prevents side effects such as hypoglycemia resulted from excess administration of the insulin. More detailed explanation of the infusion of the insulin is as follows. The base mode continuously infuses the insulin at a basic rate. The base mode is applied at a fasting in which the glucose concentration is maintained to a low level without any abrupt increase of the glucose level. The food mode infuses bulk of the insulin at a time. The food mode is used to reduce the highly concentrated glucose to a normal state after food intake. The insulin is administered by the maintenance mode in an amount of 30-50 units per day.

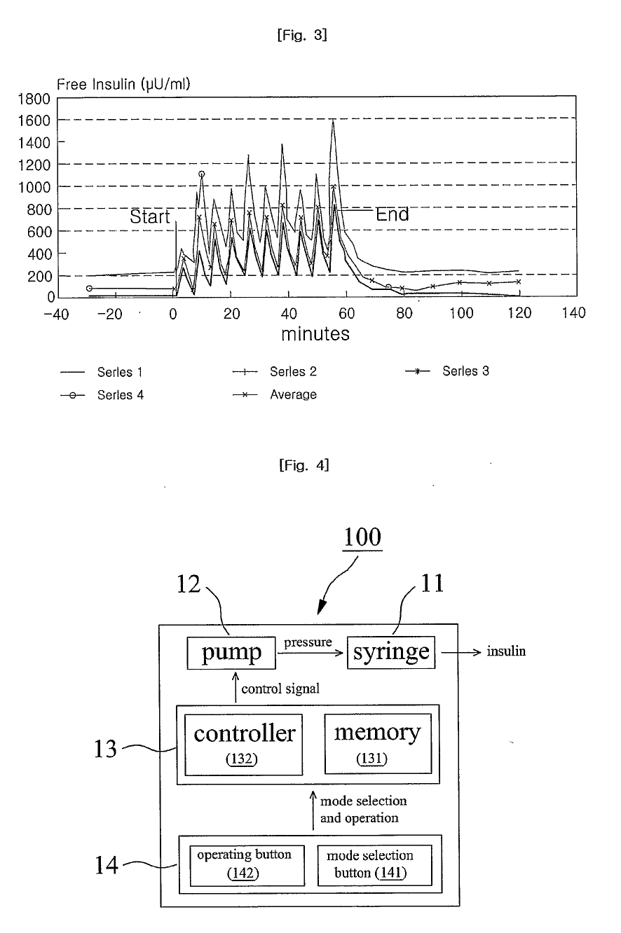

The activation mode is to restore the function of the kidney suffered from the relative deficiency of the insulin caused from the maintenance mode. The activation mode infuses the insulin in a pulsed form for a predetermined time period. For example, at the activation mode, the insulin is intravenously infused every 6-8 minutes for a period of 5-6 hours. When the insulin is infused every 6 minutes at an amount of 2 units for 6 hours, 120 units of the insulin are intravenously infused. With each of the pulsed infusions, the insulin is administered through peripheral vein at a volume of 2 units and undergoes systemic circulation. During the systemic circulation, the insulin is diluted with the blood. In order to accomplish high insulin level at the kidney, a physiological saline syringe pump supplies 1-2 mL of physiological saline solution immediately after the pulsed infusions of the insulin, which enables rapid circulation and high insulin level at the kidney. FIG. 3 is the graph showing a blood insulin profile obtained from pulsed, intravenous infusions of the insulin to induce activation of the metabolism of the kidney.

FIG. 4 is a block diagram showing a preferred embodiment of the insulin pump, in accordance with the present invention. As shown in FIG. 4, the insulin pump (100) comprises an insulin syringe (11) into which an insulin is stored, a pump (12) to pressurize the insulin syringe (11) in response to a programmed control signal, and a control device (13) that generates the programmed control signal to control the pump (12). Besides, the insulin pump (100) further comprises a selection button (14) to select various functions to be installed. The selection button (14) is comprised of a mode selection button (141) to determine an insulin injection mode; and an operating button (142) to command an insulin injection. The control device (13) is equipped with a memory (131) and a controller (132). To the memory, a control program is stored. The control program is comprised of a base mode that continuously infuses the insulin at a basic rate to control a blood glucose concentration at a fasting, a food mode that infuses the insulin at a bulk to control the blood glucose concentration after food intake, and an activation mode that intravenously infuses the insulin in pulses for a period of 5-6 hours at an amount to activate the metabolism of the kidney (typically, at an interval of one week under control of a doctor). In order to prohibit excess infusion of the insulin, a safe guard program may be further installed.

The working principle of the control program will be more fully illustrated. Insulin infusion mode is determined by the mode selection button (141). At a fasting, the mode selection button (141) will be positioned in the base mode. In this case, the control program is set to the base mode and commands to continuously infuse the insulin at a basic rate. After intake of food, the mode selection button (141) will be positioned in the food mode. The control program is set to the food mode and commands to infuse bulk of the insulin at a time. To the contrary, at the activation mode, the control program commands to infuse the insulin in pulses. The commands from the control program are delivered to the controller (132) of the control device (13), and the controller (132) produces the control signal corresponding to the selected mode to operate the pump (12).

Using the insulin pump (100), the insulin can be subcutaneously or intravenously administrated either at the base mode or at the food mode. At the activation mode, the insulin is intravenously infused in a pulsed form for a predetermined time period. Generally, the insulin pump (100) adopts short acting insulin, and this sometimes limits the application of the insulin pump (100). The short acting insulin has a very short half life (typically, 2 min). This insulin should be rapidly infused through the human body in a prescribed time limit. If not administered by the limit, the short acting insulin does not work well and the patients may suffer from hyperglycemia. Further, excess administration of the short acting insulin to avoid the shortage may result in hypoglycemia. This is fatal to the patients sensitive to the insulin. Efficient supply of the insulin is essential for the insulin pump (100).

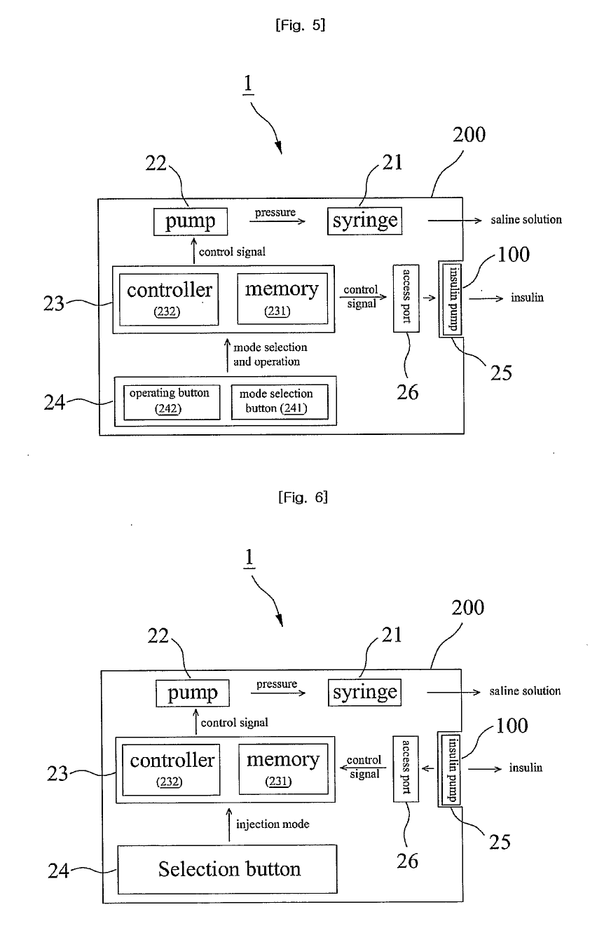

The instability of the short acting insulin administration can be avoided by the activation mode that intravenously infuses the insulin in a pulsed form. However, in a case that the infusion of the insulin is performed by the activation mode of the insulin pump (100), the insulin is infused at a low quantity and diluted with the blood. As a result, the insulin reached to the kidney does not have sufficiently high level to activate the metabolism of the kidney. In order to solve the problem, our inventor designed an insulin injection system that infuses a physiological saline solution concomitantly with the infusion of the insulin by the activation mode. FIG. 5 is a block diagram showing a preferred embodiment of the insulin injection system comprising the insulin pump, in accordance with the present invention. As shown in FIG. 5, the insulin injection system (1) of the present invention further comprises a physiological saline syringe pump (200) in addition to the insulin pump (100) mentioned in the above. The physiological saline syringe pump (200) is comprised of a physiological saline syringe (21) into which physiological saline solution is stored, a physiological saline pump (22) to pressurize the physiological saline syringe (21) in response to a programmed control signal, a control device (23) that generates the programmed control signal to control the physiological saline pump (22). Besides, the physiological saline syringe pump (200) further comprises a selection button (24) to select various functions to be installed. The physiological saline syringe pump (200) comprises the same elements with the insulin pump (100), except that the physiological saline solution, instead of the insulin, is infused at a relatively high volume. Herein, the control device (13) of the insulin pump (100) complementarily cooperates with the control device (23) of the physiological saline syringe pump (200). The physiological saline syringe pump (200) further comprises a mount (25) into which the insulin pump (100) is installed. Synchronization between the insulin pump (100) and the physiological saline syringe pump (200) is accomplished through an access port (26) mediated by electrical switching. As one exemplarily embodiment of the present invention, the control device (13) of the insulin pump (100) may be deactivated to accomplish complementary cooperation. In this case, the control device (23) of the physiological saline syringe pump (200) comprises a memory (231) to which a control program is stored and a controller (232) to generate a control signal. The control program has dual functions: one is to control the infusion of the physiological saline solution and the other is to control the infusion of the insulin. As mentioned in the above, the infusion of the insulin is controlled by one of the three modes: the base mode, the food mode and the activation mode. The user may control both the infusion of the insulin and the infusion of the physiological saline solution by manipulation of the selection button (24) of the physiological saline syringe pump (200). When the insulin infusion mode is located at the activation mode, the physiological saline syringe pump (200) infuses the physiological saline solution immediately after the infusion of the insulin. This provides rapid infusion of the insulin into the human body, thereby preventing dilution of the insulin by the blood and overcome disadvantage of the short half life.

FIG. 6 is a block diagram showing another preferred embodiment of the insulin injection system comprising the insulin pump, in accordance with the present invention. The roles of the elements in FIG. 6 are the same as shown in FIG. 5, except that the control device (13) of the insulin pump (100) is not deactivated. In this case, the insulin infusion mode is determined by the control signal from the control device (13) of the insulin pump (100). Further, when the insulin injection mode of the insulin pump (100) is located at the activation mode, the control signal is delivered from the insulin pump (100) through the access port (26) to the control device (23) of the physiological saline syringe pump (200). In response to the input of the control signal from the insulin pump (100), the control device (23) of the physiological saline syringe pump (200) generates another control signal to command the infusion of the physiological saline solution and delivers it to the physiological saline pump (22), and with the action of the physiological saline pump (22), the physiological saline solution is infused from the physiological saline syringe (21). This system simplifies the control program of the physiological saline syringe pump (200). Using the selection button (14) of the insulin pump (100), one may determine the mode of the insulin infusion and, using the selection button (24) of the physiological saline syringe pump (200), one may determine the quantity of the physiological saline solution to be infused when the insulin is infused at the activation mode.

1. An insulin pump, comprising: a) an insulin syringe into which an insulin is stored; b) a pump to pressurize the insulin syringe in response to a programmed control signal; c) a control device that generates the programmed control signal to control the pump; d) a mode selection button to determine an insulin injection mode; and e) an operating button to command an insulin injection, wherein f) the control device is equipped with a memory to store a control program and a controller to generate the programmed control signal under control of the control program, g) the control program is comprised of a base mode that continuously infuses the insulin at a basic rate to control a blood glucose concentration at a fasting, a food mode that infuses the insulin at a bulk to control the blood glucose concentration after food intake, and an activation mode that infuses the insulin in pulses for a predetermined time period, h) mode selection among the three modes is carried out by the mode selection button, i) in response to activation of the operating button, the controller of the control device delivers to the pump the programmed control signal corresponding to the selected mode, and j) with action of the pump, the insulin syringe infuses the insulin in the selected mode.

2. The insulin pump as set forth in claim 1, wherein the activation mode infuses the insulin in pulses every 6-8 minutes for a period of 5-6 hours.

3. The insulin pump as set forth in claim 1, wherein the insulin is short acting insulin.

4. An insulin injection system, comprising a physiological saline syringe pump in combination with the insulin pump of claim 1.

5. The insulin injection system as set forth in claim 4, wherein the physiological saline syringe pump comprises a′) a physiological saline syringe into which physiological saline solution is stored, b′) a physiological saline pump to pressurize the physiological saline syringe in response to a programmed control signal, c′) a control device that generates the programmed control signal to control the physiological saline pump, d′) a selection button, e′) a mount for housing the insulin pump, and f′) an access port for the synchronization with the insulin pump.

6. The insulin injection system as set forth in claim 5, wherein a control signal generated from the control device of the insulin pump is delivered to the control device of the physiological saline syringe pump and the control device of the physiological saline syringe pump is activated with aid of the control signal.

7. The insulin injection system as set forth in claim 5, wherein the access port undergoes an electrical switching when the insulin pump is installed to the physiological saline syringe pump such that the insulin pump is controlled by the control device of the physiological saline syringe pump.

8. The insulin injection system as set forth in claim 7, wherein the control device of the physiological saline syringe pump is comprised of a memory to which a control program is stored and a controller to generate a control signal, and the control program is equipped with two functions: one is to control the infusion of the insulin; and the other is to control the infusion of the physiological saline solution.
